(19) United States
(12) Patent Application Publication
(10) Pub. No.: US 2014/0213686 A1
(43) Pub. Date: Jul. 31, 2014

(54) DENTAL AUTO-MIXING METHOD, DEVICE, AND COMPOSITION FOR TEMPORARY CEMENTS

(71) Applicant: 3M INNOVATIVE PROPERTIES COMPANY, Saint Paul, MN (US)

(72) Inventors: Afshin Falsafi, Saint Paul, MN (US); Joel D. Oxman, Saint Paul, MN (US); Hoa T. Bui, Saint Paul, MN (US); Anupa N. Patel, Saint Paul, MN (US)

(21) Appl. No.: 14/242,519

(22) Filed: Apr. 1, 2014

Related U.S. Application Data
(63) Continuation of application No. 13/513,879, filed on Jun. 5, 2012.
(60) Provisional application No. 61/290,576, filed on Dec. 29, 2009.

(51) Int. Cl. A61K 6/083

(52) U.S. Cl. CPC A61K 6/083
USPC 522/181

(57) ABSTRACT

A method of dispensing a hardenable dental composition, which can form a temporary bond, comprising 1) providing multi-part hardenable dental composition comprising part (A) in the form of a paste comprising acid-reactive glass particles and a liquid selected from the group consisting of water, a monomer having at least one ethylenically unsaturated group per monomer molecule, and a combination thereof; and part (B) comprising a water soluble polyacid and a liquid selected from the group consisting of water, a monomer having at least one ethylenically unsaturated group per monomer molecule, and a combination thereof; wherein an adhesion reducing component is included in part (A); part (B); or parts (A) and (B); water is included in part (A); part (B); or parts (A) and (B); the monomer having at least one ethylenically unsaturated group per monomer molecule is included in part (A); part (B); or parts (A) and (B); and at least one component for initiating polymerization of the monomer is included in part (A); part (B); or parts (A) and (B); and 2) extruding the composition through a static mixer in fluid communication with a first reservoir containing the part (A) and a second reservoir containing the part (B); wherein a plunger is positioned in each reservoir for simultaneously forcing part (A) and part (B) into the static mixer, extruding the composition through the static mixer, and dispensing the composition; and wherein an extrusion force of less than 40 pound-force (178 newtons) according to Test Method I is applied to the plunger for extruding the composition through the static mixer without the aid of a mechanical advantage provided by an attached or external device; a method of bonding a prosthetic device to a dental structure using the dispensing method, a device for dispensing the composition using the dispensing method, a kit comprising the device, and the composition used in the dispensing method are provided.

### CROSS REFERENCE OF RELATED APPLICATIONS

This application is a Continuation Application of U.S. application Ser. No. 13/513,879 filed Jun. 5, 2012, which is a National Stage Application under 35 U.S.C. §371 of International Application Number PCT/US2010/060709 filed Dec. 16, 2010, published as WO 2011/081976 on Jul. 7, 2011, which claims the benefit of U.S. Provisional Application No. 61/290,576 filed Dec. 29, 2009, the entire contents of which are incorporated herein by reference in its entirety.

### BACKGROUND

Two-part glass ionomer cements have been in dental use for some time. Such materials are comprised of an ionic polymer component and a reactive glass component, which when mixed together in the presence of water undergo a cement setting reaction. These dental materials provide several desirable attributes including prolonged fluoride release, tolerance to moisture and saliva, good mechanical properties and excellent adhesion to dental hard tissues without pretreatments such as conditioners or adhesives. Powder-liquid, powder-paste, paste-paste, paste-liquid, and liquid-liquid two-part cements have been reported. Traditionally, the two parts have been measured out in some way and hand mixed or spatulated; although in one alternative a two-compartment capsule with pre-measured powder and liquid components has been used with vibratory mechanical mixing. Various drawbacks have become evident with these materials and methods, including, for example, mechanical strength variability, varying consistencies, unsatisfactory working or setting times, cost per application, multiple dispensing and mixing steps, mechanical mixing equipment and waste.

More recently, the use of auto mixing delivery systems for two component paste/paste dental materials has addressed some of the above limitations, providing some ease of use, time savings and consistent product performance. In the case of glass ionomer cements, such systems have included a device requiring the combination of a cartridge and a device providing a significant mechanical advantage.

However, there continues to be a growing interest in alternative methods and compositions for delivering glass ionomer cements and related materials in a faster, easier and/or more simplified manner.

### SUMMARY

It has now been found that certain multi-part hardenable glass ionomer dental compositions for preparing temporary dental cements can be dispensed through a static mixer by applying only hand pressure without the aid of a mechanical advantage provided by an attached or external device. The low force required for dispensing the compositions and small size of a dispensing device that can be used with the compositions allow, among other benefits, direct dispensing of the composition in the mouth.

Accordingly, in one embodiment, there is provided a method of dispensing a hardenable dental composition, which can form a temporary dental cement, comprising:

providing multi-part hardenable dental composition comprising:
part (A) in the form of a paste comprising:
acid-reactive glass particles and a liquid selected from the group consisting of water, a monomer having at least one ethylenically unsaturated group per monomer molecule, and a combination thereof; and
part (B) comprises:
a water soluble polyacid and a liquid selected from the group consisting of water, a monomer having at least one ethylenically unsaturated group per monomer molecule, and a combination thereof;

wherein:
an adhesion reducing component is included in part (A); part (B); or parts (A) and (B);
water is included in part (A); part (B); or parts (A) and (B);
the monomer having at least one ethylenically unsaturated group per monomer molecule is included in part (A); part (B); or parts (A) and (B); and
at least one component for initiating polymerization of the monomer is included in part (A); part (B); or parts (A) and (B);

extruding the composition through a static mixer in fluid communication with a first reservoir containing the part (A) and a second reservoir containing the part (B);

wherein a plunger is positioned in each reservoir for simultaneously forcing part (A) and part (B) into the static mixer, extruding the composition through the static mixer, and dispensing the composition; and

wherein an extrusion force of less than 40 pound-force (178 newtons) according to Test Method I is applied to the plunger for extruding the composition through the static mixer without the aid of a mechanical advantage provided by an attached or external device.

In another embodiment, there is provided a method of bonding a prosthetic device to a dental structure comprising:

dispensing the hardenable dental composition according to claim 1 onto a surface of a dental prosthetic device, a surface of a dental structure, or a combination thereof;

positioning the device on the dental structure; and

hardening the dental composition to form a temporary dental cement;

wherein the prosthetic device is selected from the group consisting of a crown, bridge, inlay, onlay, post, abutment, veneer, and prosthetic tooth; and

wherein the dental structure is a prepared tooth or an implant.

In another embodiment, there is provided a dental device comprising:

a multi-part hardenable dental composition, which can form a temporary dental cement, comprising:
part (A) in the form of a paste comprising:
acid-reactive glass particles and a liquid selected from the group consisting of water, a monomer having at least one ethylenically unsaturated group per monomer molecule, and a combination thereof; and
part (B) comprises:
a water soluble polyacid and a liquid selected from the group consisting of water, a monomer having at least one ethylenically unsaturated group per monomer molecule, and a combination thereof;

wherein:
an adhesion reducing component is included in part (A); part (B); or parts (A) and (B);
water is included in part (A); part (B); or parts (A) and (B);
the monomer having at least one ethylenically unsaturated group per monomer molecule is included in part (A); part (B); or parts (A) and (B); and
at least one component for initiating polymerization of the monomer is included in part (A); part (B); or parts (A) and (B);

a first reservoir containing the part (A);

a second reservoir containing the part (B);

a static mixer in fluid communication with or which can be connected in fluid communication with the first and second reservoirs; and

a plunger positioned in each reservoir for forcing part (A) and part (B) into the static mixer, extruding the composition through the static mixer, and dispensing the composition;

wherein an extrusion force of less than 40 pound-force (178 newtons) according to Test Method I is required for extruding the composition through the static mixer without the aid of an attached or external device for providing a mechanical advantage.

In another embodiment, there is provided a dental kit comprising the above device and a plurality of static mixers adapted for fluid communication with the first and second reservoirs.

In another embodiment, there is provided a multi-part hardenable dental composition comprising:

part (A) in the form of a paste comprising:
acid-reactive glass particles and a liquid selected from the group consisting of water, a monomer having at least one ethylenically unsaturated group per monomer molecule, and a combination thereof; and part (B) comprises:
a water soluble polyacid and a liquid selected from the group consisting of water, a monomer having at least one ethylenically unsaturated group per monomer molecule, and a combination thereof;

wherein:
an adhesion reducing component is included in part (A); part (B); or parts (A) and (B);
water is included in part (A); part (B); or parts (A) and (B);
the monomer having at least one ethylenically unsaturated group per monomer molecule is included in part (A); part (B); or parts (A) and (B); and
at least one component for initiating polymerization of the monomer is included in part (A); part (B); or parts (A) and (B);

wherein the composition can form a temporary dental cement;

wherein the composition can be extruded through a static mixer in fluid communication with a first reservoir containing the part (A) and a second reservoir containing the part (B);

wherein a plunger is positioned in each reservoir for simultaneously forcing part (A) and part (B) into the static mixer and extruding the composition through the static mixer; and

wherein an extrusion force of less than 40 pound-force (178 newtons) according to Test Method I is applied to the plunger for extruding the composition through the static mixer without the aid of a mechanical advantage provided by an attached or external device.

### DEFINITIONS

The term “temporary cement” refers to a cement which can hold a dental material, such as a dental prosthetic device, for example, a crown, in place on a dental structure, such as a prepared tooth or implant, under normal oral conditions of use for the service life of the device, and further, which facilitates easy removal of the device when needed without damage to the tooth or implant. Such temporary cements, therefore, allow easy removal, whether they are in use for a long period of time, which may be considered permanent, or for a short period of time, which may be considered temporary.

The term “temporary bond” refers to a bond formed by the temporary cement to dentin and/or enamel, such that a dental material so bonded to a dental structure can be removed easily without damage to the dental structure, such as a tooth or an implant.

The term “water soluble” refers to a material, such as a monomer or polymer, which dissolves partially or fully in water and dissolves in water alone in an amount of at least 5 g per liter of water at 25° C., or which dissolves in water combined with a monomer, a cosolvent, and/or a surfactant in an amount of at least 5 percent by weight at 25° C. The term “comprising” and variations thereof (e.g., comprises, includes, etc.) do not have a limiting meaning where these terms appear in the description and claims.

As used herein, “a,” “an,” “the,” “at least one,” and “one or more” are used interchangeably, unless the context clearly dictates otherwise.

Also herein, the recitations of numerical ranges by endpoints include all numbers subsumed within that range (e.g., the range of viscosity ratios 1:0.06 to 1:13 includes 1:0.06 to 1:13, 1:0.1 to 1:13, 1:0.25 to 1:13, 1:0.5 to 1:13, 1:0.6 to 1:13, 1:1 to 1:13, 1:0.06 to 1:10, 1:0.06 to 1:7.5, 1:0.06 to 1:5, 1:0.06 to 1:3.5, 1:0.06 to 1:1, 1:0.1 to 1:10, 1:0.25 to 1:7.5, 1:0.5 to 1:5, 1:0.6 to 1:3.5, 1:0.75 to 1:2, 1:0.9 to 1:1.1, etc.).

The above summary of the present invention is not intended to describe each disclosed embodiment or every implementation of the present invention. The description that follows more particularly exemplifies illustrative embodiments.

### BRIEF DESCRIPTIONS OF THE FIGURE

FIG. 1 is a perspective view of an assembled dental device for mixing and dispensing a multi-part hardenable dental composition as described herein.

FIG. 2 is a perspective view of a static mixer included in the assembled dental device of FIG. 1.

FIG. 3 is an exploded view in perspective of an alternative dental device for mixing and dispensing a multi-part hardenable dental composition as described herein.

FIG. 4 is a cross-sectional view of the device of FIG. 3 in assembled form showing parts (A) and (B) in separate reservoirs prior to being forced into the static mixer.

### DETAILED DESCRIPTION OF ILLUSTRATIVE EMBODIMENTS OF THE INVENTION

As indicated above, previous methods for auto mixing glass ionomer cements have included a device for providing a mechanical advantage. Examples of such devices include dispensing guns and appliers such as GC FujiCEM automix and Paste Pak Dispenser. This has been found to be undesirable, for example, because of the significant bulk required for the mechanical-advantaging device, making direct dispensing at a dental structure in the mouth difficult and/or impractical. The methods, devices, kits, and compositions presently provided allow effective static mixing and dispensing of multi-part hardenable dental compositions, which can be used for preparing temporary cements, using hand pressure without mechanical-advantaging devices. As a result, the practitioner may now conduct auto mixing of multi-part hardenable dental compositions, including glass ionomer cements, using a small sized dispensing device and without hand fatigue or exceptional hand strength.

The cements prepared using the methods, devices, compositions, and kits described herein, in certain embodiments, have been found to provide additional benefits, including ease of removing excess cement during placement and fluoride ion release. At the same time a desirable balance of retention and removal characteristics are achieved.

The methods, devices, compositions, and kits presently provided are applicable to multi-dose and unit-dose applications. In multi-dose applications, a replacement static mixer is used with each successive application of the composition. The above kit embodiment, therefore, includes a plurality of static mixers.

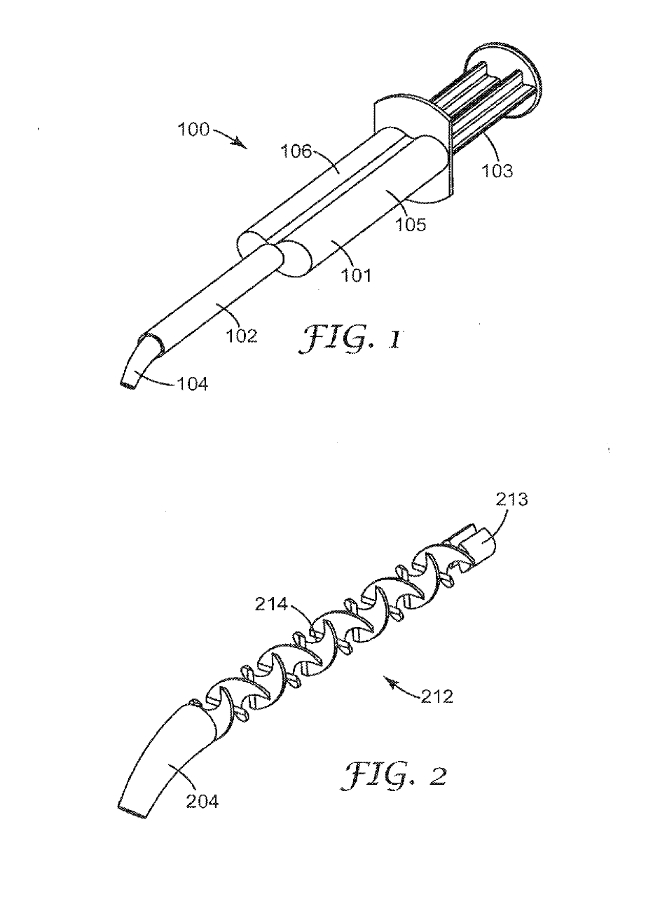

The above device described herein may be provided with a static mixer in fluid communication with the first and second reservoirs or with the static mixer not yet attached, but which can be connected in fluid communication with the first and second reservoirs at an appropriate time. FIG. 1 illustrates one example of an assembled dental device 100 in the form of a double syringe for mixing and dispensing a multi-part hardenable dental composition. Syringe body 101 includes reservoir 105, containing one part of the composition, for example, part (A), and reservoir 106, containing another part of the composition, for example, part (B). Mixing tube 102 contains a static mixer (not shown) and is equipped with optional curved dispensing tip 104. Alternatively, tube 102 may simple taper to a smaller diameter. Mixing tube 102 may be an integral part of syringe body 101, for example, when a unit-dose application is contemplated. Alternatively, mixing tube 102 may be removable and replaceable, for example, when multi-dose applications are to be carried out. Plunger 103 in device 100 is used to force parts (A) and (B) into and through mixing tube 102. As described above, only hand pressure is required to carry this out. It has now been found that an extrusion force of less than 40 pound-force (178 newtons) according to Test Method I described below meets this requirement. For certain embodiments, preferably an extrusion force of not more than 30 pound-force (133 newtons), more preferably not more than 20 pound-force (89 newtons), is required.

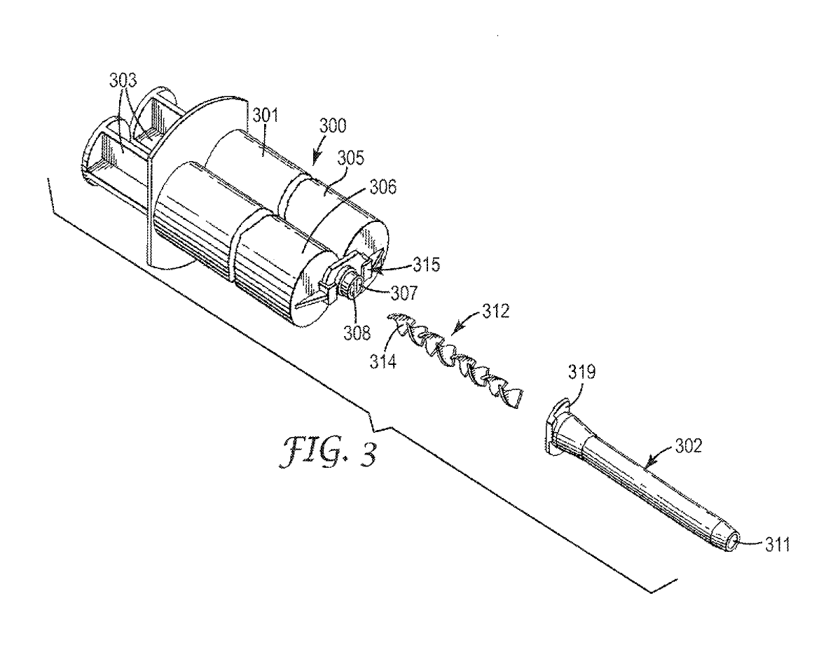

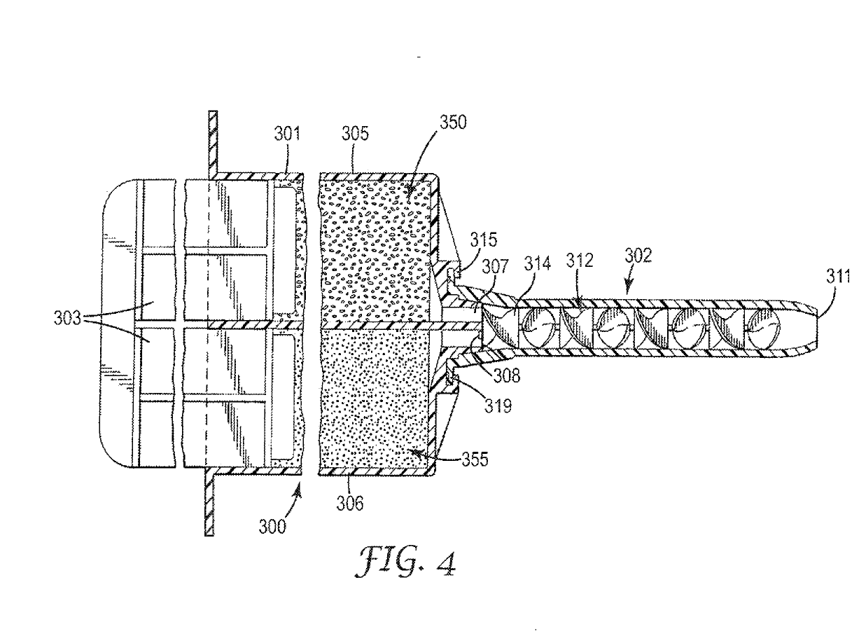

FIG. 2 illustrates static mixer 212 with ten mixing elements 214. In order to achieve adequate and reproducible mixing of parts (A) and (B), a sufficient number of mixing elements are included. For certain embodiments, including any one of the above method, device, kit, and composition embodiments, preferably the static mixer includes at least 8 mixing elements or at least 10 mixing elements. For certain of these embodiments, the static mixer includes at least 12 mixing elements. While more mixing elements may be used, the number is kept to that which is necessary for adequate and reproducible mixing, so as to prevent unnecessary back pressure resulting from additional, but unnecessary mixing elements. Static mixer 212 is also shown with optional curved dispensing tip 204 and optional closure plug 113, which can function to close outlet openings (not shown) of reservoirs 105 and 106 of device 100 in FIG. 1 to prevent contact between parts (A) and (B) when not in use.

In another example of a device described herein, FIGS. 3 (exploded view in perspective) and 4 (cross-sectional view) illustrate device 300, also in the form of a double syringe, for mixing and dispensing the multi-part hardenable dental composition. Syringe body 301 includes reservoir 305, containing part (A) 350 of the composition, and reservoir 306, containing part (B) 355 of the composition. Mixing tube 302 contains static mixer 312 with mixing elements 314 and is equipped with outlet 311. Mixing tube 302 is removable and replaceable for multi-dose applications. When mixing tube 302 is installed on syringe body 301, locking ramps 319 of mixing tube 302 are retained by locking tabs 315. Plunger 303 in device 300 is used to force parts (A) 350 and (B) 355 through exit passages 307 and 308 into and through mixing tube 302 with a relatively low force as described above.

Additional examples of specific device constructions, which may be used herein, are described, for example, in U.S. Pat. No. 4,538,920 (Drake) and U.S. Publication No. 2007/016660 A1 (Peuker et al.).

The multi-part hardenable dental compositions described herein not only provide a low extrusion force when mixed and dispensed according to the above methods and in the above described device embodiments, but also provide sufficient strength for temporarily or permanently cementing a prosthetic device to a dental structure. For certain embodiments, including any one of the above method, device, kit, and composition embodiments, when part (A) is mixed with part (B) and the mixture hardened, Shear Bond Strength according to Test Method II (described below) of the resulting hardened cement is greater than 0.2 MPa. For certain of these embodiments, preferably the Shear Bond Strength is at least 0.5 MPa. In order to facilitate removal, the hardened cement has a Shear Bond Strength of less than 2 MPa, preferably not more than 1 MPa. These bond strength values refer to bond strengths to either dentin or enamel.

For certain embodiments, preferably each part of the multi-part hardenable dental compositions described herein includes a balance of components for ease of compatiblizing each part with the other during mixing. Accordingly, for certain embodiments, including any one of the above embodiments, part A comprises the acid-reactive glass particles, and a water soluble liquid monomer having at least one ethylenically unsaturated group per monomer molecule; water; and the adhesion reducing component; and part B comprises the polyacid; and a liquid monomer having at least one ethylenically unsaturated group per monomer molecule. For certain of these embodiments, preferably at least one of the parts of the multi-part hardenable composition includes a component that provides some cross linking in the composition when hardened. For certain of these embodiments, preferably part B further comprises a liquid monomer having at least two ethylenically unsaturated groups per monomer molecule and having a viscosity less than or equal to the viscosity of Bis-GMA (2,2-bis[4-(2-hydroxy-3-methacryloyloxypropoxy)phenyl]propane, CAS No. 1565-94-2, [H₂C═CH(CH₃)CO₂CH₂CH(OH)CH₂OC₆H₄-4-]₂C(CH₃)₂). For certain of these embodiments, the liquid monomer has a viscosity of at most 50 percent of the viscosity of Bis-GMA.

Each part of the multi-part hardenable dental compositions described herein has a viscosity which is balanced with respect to the other parts of the composition. For certain embodiments, preferably the viscosity of each part is less than 20 fold higher or lower than that of any other part of the composition. For certain embodiments, including any one of the above embodiments, part (A) and part (B) each independently have a viscosity not less than 6 pascal-second (Pa·s) and not greater than 100 Pa·s. For certain of these embodiments, the ratio of part (B) to part (A) viscosity is 1:0.06 to 1:13. For certain of these embodiments, preferably the ratio of part (B) to part (A) viscosity is 1:0.6 to 1:3.5, more preferably 1:0.9 to 1:1.6.

It has been found that the viscosity of part (A) can be controlled for low extrusion force using coarse particles of the acid-reactive glass. The coarse particles have an average particle diameter of greater than about 2 to about 30 micrometers. For certain of these embodiments, preferably the coarse particles have an average particle diameter of not more than about 20 micrometers. For certain of these embodiments, the coarse particles have an average particle diameter of 3 to 10 micrometers.

In order to achieve good mixing and low extrusion force as described above and, at the same time, balanced strength properties (e.g., sufficient strength for desired durability and ease of removal), for certain embodiments, including any one of the above embodiments, the acid-reactive glass particles are present in part (A) in an amount of 30 to 70 weight percent. For certain of these embodiments, preferably the acid-reactive glass particles are present in part (A) at 40 to 60 weight percent.

For certain embodiments, including any one of the above embodiments, part (A) includes water. This provides further control of the viscosity of part (A) and may further increase compatibility with other parts of the composition for good mixing. For certain of these embodiments, the amount of water in part (A) is 5 to 20 percent by weight based upon the total weight of part (A).

Nonreactive fillers may also be included in the compositions described herein to control viscosity as well as for other reasons, such as to achieve a desired appearance, impart desired strength properties, impart radiopacity, and the like. For certain embodiments, including any one of the above embodiments, part (A), part (B), or part (A) and part (B) further include a nonreactive filler in an amount of 1 to 50 weight percent based upon the total weight of the part which includes the nonreactive filler.

Non-reactive fillers may be selected from one or more of any material suitable for incorporation in compositions used for medical applications, such as fillers currently used in dental restorative compositions and the like. The filler preferably has a maximum particle diameter less than about 50 micrometers and an average particle diameter less than about 10 micrometers. When the present compositions are used as a luting cement, the filler is finely divided and has a maximum particle diameter less than about 15 micrometers in order to provide a luting cement with a film thickness in accordance with ISO Standard 3107 of less than about 25 micrometers. The filler can have a unimodal or polymodal (e.g., bimodal) particle size distribution. For certain embodiments, including any one of the above embodiments which includes a nonreactive filler, the nonreactive filler is selected from the group consisting of inorganic material, crosslinked organic material, and a combination thereof. Suitable crosslinked organic materials are insoluble in the composition, and are optionally filled with inorganic filler. The filler should be non-toxic and suitable for use in the mouth. The filler can be radiopaque, radiolucent or non-radiopaque.

Examples of suitable non-reactive inorganic fillers are naturally-occurring or synthetic materials such as quartz, nitrides (e.g., silicon nitride), glasses derived from, for example, Ce, Sb, Sn, Zr, Sr, Ba and Al, colloidal silica, colloidal zirconia, feldspar, borosilicate glass, kaolin, talc, titania, and zinc glass; low Mohs hardness fillers such as those described in U.S. Pat. No. 4,695,251; and submicron silica particles (e.g., pyrogenic silicas such as the “Aerosil” Series “OX 50”, “130”, “150” and “200” silicas sold by Degussa and “Cab-O-Sil M5” silica sold by Cabot Corp.); metallic powders such as those disclosed in U.S. Pat. No. 5,084,491, especially those disclosed at column 2, lines 52-65; and combinations thereof.

Examples of suitable non-reactive organic filler particles include filled or unfilled pulverized polycarbonates, polyepoxides, and the like. Preferred non-reactive filler particles are quartz, submicron silica and zirconia, and non-vitreous microparticles of the type described in U.S. Pat. No. 4,503,169. Mixtures of these non-reactive fillers are also contemplated, as well as combination fillers made from organic and inorganic materials.

For certain embodiments, including any one of the above embodiments which includes a nonreactive filler, preferably the nonreactive filler is selected from the group consisting of fumed silica, zirconia-silica, quartz, nonpyrogenic silica, and a combination thereof.

The surface of the non-reactive filler particles, in certain embodiments, preferably is treated with a coupling agent in order to enhance the bond between the filler and polymerizable components when the composition is hardened. The use of suitable coupling agents include gamma-methacryloxypropyltrimethoysilane, gamma-mercaptopropyltriethoxysilane, gamma-aminopropyltrimethoxysilane, SILQUEST A-1230 (Momentive Performance Chemicals), and the like.

For certain embodiments, including any one of the above embodiments which includes a nonreactive filler, part (B) includes the nonreactive filler in an amount of 5 to 45 weight percent based upon the total weight of part (B). For certain of these embodiments, preferably the nonreactive filler is selected from the group consisting of fumed silica, zirconia-silica, quartz, nonpyrogenic silica, and a combination thereof. For certain of these embodiments, preferably the nonreactive filler is silane treated zirconia-silica.

Part (B) may be in the form of a viscous liquid, a gel, or a paste. The viscous liquids and the gels typically contain relatively lower amounts or no nonreactive filler. The pastes typically include relatively larger amounts of nonreactive filler. For certain embodiments, including any one of the above embodiments, part (B) is in the form of a paste.

As indicated above, the multi-part hardenable compositions described herein include a liquid monomer having at least one ethylenically unsaturated group per monomer molecule, and in certain embodiments, preferably such monomers are partially or fully water soluble. For certain embodiments, preferably ethylenically unsaturated groups include allyl, vinyl, acrylate, and methacrylate groups. For certain embodiments, such monomers have a relatively low molecular weight and include only one ethylenically unsaturated group per monomer molecule. For certain embodiments, preferably the molecular weight of such monomers is about 100 to about 1000. For certain embodiments, including any one of the above embodiments which includes a water soluble liquid monomer in the multi-part hardenable composition, the monomer is selected from the group consisting of 2-hydroxyethyl methacrylate, glycerol monomethacrylate, polyethylene glycol dimethacrylate, sorbitol methacrylate, and a combination thereof.

Such monomers have been found to contribute to the ease of compatibilizing each part with the other during auto mixing and achieving the desired viscosity described above for parts (A) and (B).

Suitable water soluble polyacids for part (B) include, but are not limited to, homo- or copolymers of unsaturated mono-, di-, and tricarboxylic acids, for example, homo- or copolymers of acrylic acid, itaconic acid and maleic acid. For certain embodiments, preferably the water soluble polyacid comprises a polymer having sufficient pendent ionic groups to undergo a setting reaction in the presence of a reactive filler and water, and sufficient pendent non-ionically polymerizable groups to enable the resulting mixture to be cured by a redox curing mechanism and/or by exposure to radiant energy.

For certain embodiments, including any one of the above embodiments, the polyacid is of the Formula I:

B(X)ₘ(Y)ₙ I

wherein B is an organic backbone, each X independently is an ionic group which can undergo a setting reaction in the presence of water and the acid-reactive glass particles, each Y independently is a non-ionically polymerizable group, m is at least 2, and n is at least 1. For certain of these embodiments, X is —COOH and Y is an ethylenically unsaturated group. For certain of these embodiments, preferably the backbone B is an oligomeric or polymeric backbone of carbon-carbon bonds, optionally containing non-interfering substituents such as oxygen, nitrogen or sulfur heteroatoms. The term “non-interfering” refers to substituents or linking groups that do not unduly interfere with either the ionic or the non-ionic polymerization reaction. For certain of these embodiments, preferably B is a hydrocarbon backbone. X and Y groups can be linked to the backbone B directly or by means of any non-interfering linking group, such as substituted or unsubstituted alkylene, alkyleneoxyalkylene, arylene, aryleneoxyalkylene, alkyleneoxyarylene, arylenealkylene, or alkylenearylene groups. Alkylene and arylene refer to the divalent forms of alkyl and aryl, respectively. The linking group may also include linkages such as —OC(═O)—, —C(═O)NH—, —NH—C(═O)O—, —O—, and the like, and combinations thereof, wherein each of these may be used in either direction. For certain of these embodiments, Y is attached to B via an amide linkage. For certain of these embodiments, preferably Y is an acryloyloxy, methacryloyloxy, acrylamido, or methacrylamido group.

The polyacid of Formula I can be prepared according to a variety of synthetic routes, including, but not limited to, (1) reacting n X groups of a polymer of the formula B(X)ₘ₊ₙ with a suitable compound in order to form n pendent Y groups, (2) reacting a polymer of the formula B(X)ₘ at positions other than the X groups with a suitable compound in order to form n pendent Y groups, (3) reacting a polymer of the formula B(Y)ₘ₊ₙ or B(Y)ₙ, either through Y groups or at other positions, with a suitable compound in order to form m pendent X groups and (4) copolymerizing appropriate monomers, e.g., a monomer containing one or more pendent X groups and a monomer containing one or more pendent Y groups. The synthetic route (1) above is preferred. Such groups can be reacted by the use of a “coupling compound”, i.e., a compound containing both a Y group and a reactive group capable of reacting with the polymer through an X group, thereby covalently linking the Y group to the backbone B in a pendent fashion. Suitable coupling compounds are organic compounds, optionally containing non-interfering substituents and/or non-interfering linking groups between the Y group and the reactive group.

Preferred polyacids of Formula I are conveniently prepared by reacting a polyalkenoic acid (e.g., a polymer of formula B(X)ₘ₊ₙ wherein each X is a carboxyl group) with a coupling compound containing both an ethylenically unsaturated group and a group capable of reacting with a carboxylic acid group. The molecular weight of the resultant ionomers is preferably between about 250 and about 500,000, and more preferably between about 1,000 and about 100,000. As referred to herein, “molecular weight” means weight average molecular weight. These polyacids are selected to be water soluble. Suitable polyalkenoic acids for use in preparing the polyacids used herein include those homopolymers and copolymers of unsaturated mono-, di-, and/or tricarboxylic acids commonly used to prepare glass ionomer cements. Representative polyalkenoic acids are described, for example, in U.S. Pat. Nos. 3,655,605; 4,016,124; 4,089,830; 4,143,018; 4,342,677; 4,360,605; and 4,376,835. Preferred polyalkenoic acids are those prepared by the homopolymerization and copolymerization of unsaturated aliphatic carboxylic acids, for example acrylic acid, 2-chloroacrylic acid, 3-chloroacrylic acid, 2-bromoacrylic acid, 3-bromoacrylic acid, methacrylic acid, itaconic acid, maleic acid, glutaconic acid, aconitic acid, citraconic acid, mesaconic acid, fumaric acid and tiglic acid. Suitable monomers that can be copolymerized with the unsaturated aliphatic carboxylic acids include unsaturated aliphatic compounds such as acrylamide, acrylonitrile, vinyl chloride, allyl chloride, vinyl acetate, and 2-hydroxyethyl methacrylate (“HEMA”). Ter- and higher polymers may be used if desired. For certain embodiments, preferably the homopolymers and copolymers of acrylic acid are used. The polyalkenoic acid should be substantially free from unpolymerized monomers and other undesirable components. For certain embodiments, preferably the polyalkenoic acids include polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof.

Polymers of formula B(X)ₘ₊ₙ can be prepared by copolymerizing an appropriate mixture of monomers and/or comonomers. Preferably, such polymers are prepared by free radical polymerization, e.g., in solution, in an emulsion, or interfacially. Such polymers can be reacted with coupling compounds in the presence of appropriate catalysts.

As indicated above, coupling compounds suitable for preparing polyacids for use herein include compounds that contain at least one group capable of reacting with X in order to form a covalent bond, as well as at least one polymerizable ethylenically unsaturated group. When X is carboxyl, a number of groups are capable of reacting with X, including both electrophilic and nucleophilic groups. Examples of such groups include hydroxyl, amino, isocyanato, halo carboxyl, and oxiranyl. Examples of suitable coupling compounds include, but are not limited to, acryloyl chloride, methacryloyl chloride, vinyl azalactone, allylisocyanate, 2-hydroxyethyl methacrylate, 2-aminoethyl methacrylate, and 2-isocyanatoethyl methacrylate. Other examples of suitable coupling compounds include those described in U.S. Pat. Nos. 4,035,321 and 5,814,682, the disclosures of which are hereby incorporated by reference.

For certain embodiments, including any one of the above embodiments, the polyacid is selected from the group consisting of the reaction product of a polymer selected from the group consisting of polyacrylic acids, copolymers of acrylic and itaconic acids, copolymers of acrylic and maleic acids, copolymers of methyl vinyl ether and maleic anhydride or maleic acid, copolymers of ethylene and maleic anhydride or maleic acid, copolymers of styrene and maleic anhydride or maleic acid, and a combination thereof with a coupling compound selected from the group consisting of acryloyl chloride, methacryloyl chloride, vinyl azalactone, allylisocyanate, 2-hydroxyethyl methacrylate, 2-aminoethyl methacrylate, and 2-isocyanatoethyl methacrylate.

For certain embodiment, including any one of the above embodiments which includes a liquid monomer having at least two ethylenically unsaturated groups per monomer molecule, the polyacid is insoluble in the monomer having at least two ethylenically unsaturated groups per monomer molecule. Insoluble means that less than 3% by weight polyacid dissolves in the monomer at 25° C. For certain of these embodiments, the monomer having at least two ethylenically unsaturated groups per monomer molecule is selected from the group consisting of urethane dimethacrylate, glycerol dimethacrylate, triethyleneglycol dimethacrylate, polyethyeneglycol dimethacrylates, and a combination thereof.

Suitable acid-reactive glass includes ion-leachable glasses, e.g., as described in U.S. Pat. Nos. 3,655,605; 3,814,717; 4,143,018; 4,209,434; 4,360,605 and 4,376,835. For certain embodiments, the acid-reactive glass is preferably selected from borate glasses, phosphate glasses and fluoroaluminosilicate glasses. For certain embodiments, including any one of the above embodiments, preferably the acid-reactive glass is fluoroaluminosilicate (FAS) glass. Suitable acid-reactive glasses are also available from a variety of commercial sources familiar to those skilled in the art. For example, suitable acid-reactive glasses can be obtained from a number of commercially available glass ionomer cements, such as “GC Fuji LC” cement and “Kerr XR” ionomer cement. Mixtures of acid-reactive glasses can be used if desired.

The acid-reactive glass particles may also be subjected to a surface treatment. Suitable surface treatments include acid washing, treatment with phosphates, treatment with chelating agents such as tartaric acid, treatment with a silane or silanol coupling agent. For certain embodiments, preferably the acid-reactive glass particles are silanol treated fluoroaluminosilicate glass particles, as described in U.S. Pat. No. 5 5,332,429, the disclosure of which is incorporated herein by reference.

When mixing the parts of the multi-part composition, it has been found that better mixing occurs when the parts are in a volume ratio approaching or at about 1:1, as compared with using relatively larger differences in the volumes. This results in better properties of the composition when hardened, for example, higher shear bond strength and/or diametral tensile strength (DTS). Moreover, mixing parts in volumes that are very different from each other increases the possibility of introducing error into the amounts of the components, which would adversely affect properties. Accordingly, for certain embodiments, including any one of the above embodiments, the part (A) and the part (B) are in a volume ratio of 1.2:1 to 1:1.2.

As indicated above, an extrusion force of less than 40 pound-force (178 newtons) according to Test Method I applied to the plunger for extruding the present composition through the static mixer can now be carried out without the aid of a mechanical advantage provided by an attached or external device. Extrusion forces considerably lower than 178 newtons have now been achieved. For certain embodiments, including any one of the above embodiments, the force is less than 30 pound-force (133 newtons). For certain of these embodiments, the force is less than 20 pound-force (89 newtons), preferably less than 15 pound-force (67 newtons). For certain of these embodiments, the force is 10 to 15 pound-force (44 to 67 newtons). It is noted that stiction can make dispensing the composition with an even lower extrusion force, such as an extrusion force of 5 pound-force or less, undesirable. This is because the plunger may momentarily stick, and overcoming this stiction may require less force than that required to dispense the composition, resulting in an uncontrolled amount of composition being dispensed.

As indicated above, the multi-part hardenable dental compositions described herein include one or more adhesion reducing components. The adhesion reducing component comprises one or more materials that are present in sufficient quantity to provide a temporary cement when the composition is hardened such that the cement has a shear bond strength of less than 2.0 MPa. Adhesion reducing components include, but are not limited to, salts or bases to partially neutralize the polyacid, non-acid reactive materials to reduce the proportion of acid-reactive species in the composition, and substitution of some or all of the polyacid with a polyacid of lower molecular weight. The adhesion reducing component may be added to one or more of the parts of the multi-part compositions and may be added in such amount as to provide a cement when the composition is hardened having a shear bond strength in the range desired for its intended purpose. Mixtures of the adhesion reducing components may be utilized.

Suitable salts or bases that can be used to partially neutralize the polyacid of the composition generally include salts or bases wherein the pKa of the conjugate acid of the salt is greater than the pKa of the polyacid. Preferred salts and bases are sodium citrate, potassium phosphate, monoammonium phosphate, sodium hydroxide, potassium hydroxide, lithium, sodium or potassium salts, magnesium oxide, sodium oleate, hydrated or non-hydrated sodium phosphates and hydrated or non-hydrated potassium phosphates. Typically, the salt or base will be present in about 0.001 to about 10 weight %, preferably from about 0.5 to about 5 weight %, based on the total weight of the cement composition.

Suitable non-acid reactive materials include any or all of the non-reactive fillers mentioned above, either alone or in combination. Suitable non-acid reactive materials also include chelating agents such as tartaric acid. Suitable non-acid reactive materials further include water, polyhydric alcohols such as glycerol, poly(ethylene glycol) and poly(propylene glycol), poly(vinyl acetate) and non-acid reactive monomers, polymers and oligomers, e.g., polyethylene glycol dimethacrylate, glycerol dimethacrylate, Bis-GMA, triethylene glycol dimethacrylate, 2-hydroxyethyl methacrylate, polypropylene glycol dimethacrylate, urethane dimethacrylate and other non-acid reactive resins suitable for incorporation into conventional dental materials. The non-acid reactive material is present in the multi-part hardenable composition in an amount of 1 to 95 weight %, preferably from 10 to 80 weight %, based on the total weight of the cement composition.

For certain embodiments, including any one of the above embodiments, preferably the adhesion reducing component is a non-acid reactive material selected from the group consisting of zirconia:silica microparticles, submicron silica, water, glycerol, poly(ethylene glycol), polyethylene glycol dimethacrylate, triethylene glycol dimethacrylate, 2-hydroxyethyl methacrylate, water soluble or dispersible celluloses, and a combination thereof. For certain of these embodiments, the adhesion reducing component is selected from the group consisting of polyethylene glycol, glycerol, water soluble or dispersible celluloses, and a combination thereof.

Substitution of some or all of the polyacid with a polyacid of lower molecular weight may alternatively or additionally be utilized to provide a composition, which when hardened is a temporary cement with sufficiently low shear bond adhesion. For example, a polyacid, e.g., polyacrylic acid, with a molecular weight of 2,000 may be used instead of a polyacid having a molecular weight of 25,000 to 40,000. Polyacids available commercially include those sold by Aldrich Chemical Co., Inc. with molecular weights of 2,000, 5,000, 90,000 and 250,000 and polyacrylic acid sold under the tradename “GOODRITE” (from BFGoodrich Co., Specialty Polymers & Chemicals Division, Cleveland, Ohio) and available in molecular weights ranging from 2,000 to 240,000. The lower molecular weight polyacids generally have a lower solids content. When desiring to formulate a paste incorporating these lower molecular weight polyacids, the polyacids can be concentrated without undesirable gellation to achieve a solids content equivalent to or higher than a commercially available higher molecular weight polyacid. Typically, the polyacid of lower molecular weight will be present in about 2 to about 40 weight %, preferably from about 3 to about 20 weight %, based on the total weight of the composition.

The multi-part hardenable dental composition used in the embodiments described herein include at least one component for initiating polymerization of the monomers in the composition and thereby further harden and strengthen the composition to a level greater than that provided by the ionic setting reaction, which occurs between the acid-reactive glass particles and the polyacid. For certain embodiments, including any one of the above embodiments, the multi-part hardenable dental composition can undergo hardening by heat or light activated polymerization or redox polymerization. For certain of these embodiments, the multi-part hardenable dental composition can undergo hardening by photopolymerization or redox polymerization.

Redox polymerization is provided by separately incorporating an oxidizing agent and a reducing agent as a redox catalyst system into the dental composition for curing via a redox reaction. Various redox systems and their use in ionomer cements are described in U.S. Pat. No. 5,154,762, the disclosure of which is incorporated herein by reference. A metal complexed ascorbic acid is a preferred reducing agent that provides cure with excellent color stability. This reducing agent and redox system is more fully described in U.S. Pat. No. 5,501,727, the disclosure of which is incorporated herein by reference. The oxidizing agent should react with or otherwise cooperate with the reducing agent to produce free radicals capable of initiating polymerization of the ethylenically unsaturated groups. The preferred amount for each of the reducing agent and the oxidizing agent is about 0.01 to about 10%, more preferably about 0.02 to about 5%, based on the total weight (including water) of the unset composition.

The oxidizing agent and the reducing agent preferably are sufficiently shelf stable and free of undesirable coloration to permit their storage and use under typical dental conditions. The oxidizing agent and the reducing agent are sufficiently soluble and present in an amount sufficient to permit an adequate free radical reaction rate. This can be evaluated by combining all of the ingredients of the cement except for the filler under safelight conditions and observing whether or not a hardened mass is obtained.

Suitable oxidizing agents include persulfates such as sodium, potassium, ammonium and alkyl ammonium persulfates, benzoyl peroxide, hydroperoxides such as cumene hydroperoxide, tert-butyl hydroperoxide, tert-amyl hydroperoxide and 2,5-dihydroperoxy-2,5-dimethylhexane, salts of cobalt (III) and iron (III), hydroxylamine, perboric acid and its salts, salts of a permanganate anion, and combinations thereof Hydrogen peroxide can also be used, although it may, in some instances, interfere with the photoinitiator, if one is present. The oxidizing agent may optionally be provided in an encapsulated form as described in U.S. Pat. No. 5,154,762.

Reducing agents include ascorbic acid, metal complexed ascorbic acid, aromatic amines such as dimethylaminophenethanol and dihydroxyethyl-p-toludine, cobalt (II) chloride, ferrous chloride, ferrous sulfate, hydrazine, hydroxylamine, oxalic acid, thiourea, alkyl thioureas and salts of a dithionite, 1-allyl-2-thiourea, thiosulfate, aromatic sulfinic acid salts such as benzene sulfinic salts and p-toluenesulfinic salts, sulfite anion and a combination thereof. Ascorbic acid and aromatic tertiary amines are preferred reducing agents. For certain embodiments, a secondary ionic salt may be used to enhance stability of the system, such as described in U.S. Pat. No. 6,982,288.

The ionomer cement systems of the invention may optionally contain one or more suitable initiators that act as a source of free radicals when activated by heat or light. Such initiators can be used alone or in combination with one or more accelerators and/or sensitizers. The initiator should be capable of promoting free radical polymerization and/or crosslinking of the ethylenically unsaturated moiety on exposure to light of a suitable wavelength and intensity. The initiator preferably is also sufficiently shelf stable and free of undesirable coloration to permit its storage and use under typical dental conditions. Visible light photoinitiators are preferred. The photoinitiator preferably is partially or fully soluble in the combined liquid components of the composition parts (A and B).

Free radical-generating photoinitiators may be used alone, but in certain embodiments, preferably are used in combination with a photosensitizer and/or an accelerator. Such initiators can generate free radicals for addition polymerization upon exposure to light energy having a wavelength between 200 and 800 nanometers.

Suitable photoinitiators (i.e., photoinitiator systems that include one or more compounds) include binary and ternary photoinitiators. In one example, a ternary photoinitiator may include an iodonium salt, a photosensitizer, and an electron donor compound as described in U.S. Pat. No. 5,545,676 (Palazzotto et al.). Examples of iodonium salts include diaryl iodonium salts, e.g., diphenyliodonium chloride, diphenyliodonium hexafluorophosphate, diphenyliodonium tetrafluoroborate, and tolylcumyliodonium tetrakis(pentafluorophenyl)borate. Examples of photosensitizers include monoketones and diketones that absorb some light within a range of about 400 nanometers to 520 nanometers, preferably 450 to 500 nanometers. Preferred are alpha diketones that absorb light within these ranges. Examples of such photosensitizers include camphoroquinone, benzil, furil, 3,3,6,6-tetramethylcyclohexanedione, phenanthraquinone, 1-phenyl-1,2-propanedione, and other 1-aryl-1-alkyl-1,2-ethanediones, and cyclic alpha diketones. Most preferred is camphoroquinone. Preferred electron donor compounds include substituted amines, e.g., ethyl dimethylaminobenzoate.

The photoinitiator, when utilized, should be present in an amount sufficient to provide the desired rate of polymerization. This amount will be dependent in part on the light source, the thickness of the layer to be exposed to radiant energy, and the extinction coefficient of the photoinitiator. Typically, the photoinitiator components will be present at a total weight of about 0.01 to about 5%, more preferably from about 0.1 to about 5%, based on the total weight of the composition.

Additional components, which are suitable for use in the oral environment, may optionally be used in the multi-part hardenable compositions described herein. In one example, such components include solvents, cosolvents (e.g., alcohols) or diluents. In another example, indicators, dyes, pigments, inhibitors, accelerators, viscosity modifiers, wetting agents, tartaric acid, chelating agents, surfactants, buffering agents, stabilizers (including free-radical stabilizers), submicron silica particles, additives that impart fluorescence and/or opalescence, modifying agents that prolonged working time, and other materials that will be apparent to those skilled in the art may be used. Additionally, medicaments or other therapeutic substances can be optionally added to the compositions. Examples include whitening agents, breath fresheners, flavorants, fragrances, anticaries agents (e.g., xylitol), fluoride sources, remineralizing agents (e.g., calcium phosphate compounds), enzymes, anesthetics, clotting agents, acid neutralizers, chemotherapeutic agents, immune response modifiers, thixotropes, polyols, anti-inflammatory agents, antimicrobial agents, antifungal agents, agents for treating xerostomia, desensitizers, and the like of the type which may be used in dental compositions. Combinations of any of the above additives may also be used in the compositions described herein. The selection and amount of any one such additive can be determined by one of skill in the art according to the desired result.

Modifying agents which may prolong the time between the beginning of the setting reaction in a restoration and the time sufficient hardening has occurred to allow subsequent clinical procedures to be performed on the surface of the restoration include, e.g., alkanolamines such as ethanolamine and triethanolamine, and mono-, di-, and tri-sodium hydrogenphosphates. Modifying agents can be added to either part A or part B. When used, they are present at a concentration between about 0.1 to 10 percent by weight, based on the total composition weight.

Certain stabilizers provide color stability. Such stabilizers include oxalic acid, sodium metabisulfite, sodium bisulfate, sodium thoisulfate, metaphosphoric acid, and combinations thereof.

Free radical stabilizers can be used with a photoinitiator to prevent premature polymerization or to adjust the working time in free radically initiated compositions. Suitable examples of free radical stabilizers include, e.g., butylated hydroxytoluene (BHT) and methyl ethyl hydroquinone (MEHQ).

Submicron silica particles may be used to improve the handling properties.

Suitable silica particles include pyrogenic silicas such as AEROSIL series OX 50, 130, 150, 200, and R-8125, available from Degussa Corp., and CAB-O-SIL M5 silica available from Cabot Corporation.

Viscosity modifiers include thickening agents. Suitable thickening agents include hydroxypropyl cellulose, hydroxymethyl celluose, carboxymethylcellulose and its various salts such as sodium, and combinations thereof.

The methods, devices, and compositions described herein are well suited for a number of dental applications, such as, for example, a luting cement used to anchor or hold a prosthetic device (e.g., crown, bridge, inlay, onlay, post, abutment, veneer, prosthetic tooth, and the like) in place in the mouth; a restorative or filler material used, for example, for filling a cavity; a thin film used, for example, as a liner on dentin and enamel or a sealant or sealing material on enamel; an orthodontic bracket adhesive; a band cement; and the like. For certain embodiments, including any one of the above embodiments, the multi-part hardenable dental composition is selected from the group consisting of a liner material, a luting material, a restorative material, an endodontic material, and a sealing material. In one example, a temporary crown is held in place until a final crown is available, yet the temporary crown can be readily removed when needed. In another example, a prosthetic device, such as an abutment or crown, is held in place on an implant where ease of retrieving and/or maintaining the implant over its life of service is desired. For certain embodiments, the multi-part hardenable dental composition is an orthodontic bracket adhesive material or band cement.

Objects and advantages of this invention are further illustrated by the following examples, but the particular materials and amounts thereof recited in these examples, as well as other conditions and details, should not be construed to unduly limit this invention.

### EXAMPLES

### Abbreviations, Descriptions, and Sources of Materials

| Abbrevi- | Description and Source of Material (Unless otherwise |
|---|---|
| ation | indicated, available from Sigma-Aldrich, St. Louis, MO.) |
| CPQ | Camphorquinone |
| ATU | Allyl thiourea |
| BPO | Benzoyl peroxide |
| BHT | 2,6-di-tert-butyl-4-methylphenol |
| DMAPE | 4-dimethylaminophenethyl alcohol |
| DI Water | Deionized water |
| Zr—Si | Silane treated zirconia-silica filler prepared as |
| Filler | described in U.S. Pat. No. 6,818,682 at col. 11, |
| | line 41 through col. 12, line 10 |
| HEMA | 2-hydroxyethyl methacrylate |
| HPC | Hydroxypropyl cellulose (Hercules Inc., Wilmington, DE) |
| PEG 300 | Polyethylene glycol having a weight average molecular |
| | weight of about 300 (Union Carbide, Houston, TX) |
| PEGDMA | Polyethylene glycol dimethacrylate [Mn (number |
| 400 | average molecular weight) of about 400] (Sartomer, |
| | Exton, PA) |
| PP | Potassium persulfate |
| R812S | Fumed silica (Degussa, Parsippany, NJ) |
| ST | Coarse FAS glass with silane treatment having a |
| Coarse | particle size of about 3 micrometers and treated as |
| Glass | described in U.S. Pat. No. 7,173,074, column 13 |
| | (Mo-Sci Corp., Columbia, MO) |
| UDMA | Urethane dimethacrylate (Dajac Labs, Trevose, PA) |
| VBCP | Reaction product of 2-isocyanatoethyl methacrylate |
| | and a copolymer of acrylic acid and itaconic acid |
| | prepared as described in U.S. Pat. No. 5,130,347, |
| | Example 11 |

### Dispensing Equipment

Auto mixing was carried out using a MIXPAC syringe (Sulzer Chemtech, Switzerland) with a medium auto mixing tip. The MIXPAC syringe is 5 ml syringe with dual barrels (1:1 volume ratio) for multi-dose applications. The part numbers of the syringe parts and mixing tip were as follows:

SDL X05-01-52 5 ml double syringe, black (each barrel having volume of 5 mL)
PPD X05-01-10SI 5 ml piston with silicone o-ring in each barrel
PLH X05-46 5 ml plunger
ML 2.5-12-SB 12-element mixing tip (medium)
VL 002-S1 Cap for protecting the contents of the syringe during storage

### Test Method I—Extrusion Force

Extrusion force was tested using an (Instron 1123, Instron Corp. Canton, Mass.) with a crosshead speed at 100 mm/min on the above described 5 ml MIXPAC syringe with the medium mixing tip. The MIXPAC syringe with a medium mixing tip on one end and a plunger inserted in the other end was inserted into a hole on a sample holder, so the Mixpac was held steady. As the plunger was pushed into the syringe, the peak force (extrusion force) required while pushing the plunger a distance of 14 mm into the syringe was measured in unit of pound-force (Ib-f).

### Test Method II—Compressive Strength

Compressive strength was evaluated by first injecting the auto-mixed cement samples into a glass tube having a 4 mm inner diameter. The ends of the tube were plugged with silicone plugs. The filled tubes were subjected to 0.275 MPa pressure for 5 minutes. The samples were then placed in a chamber at 37 degree C. and 90% relative humidity and allowed to stand for 1 hour. The cured samples were next placed in 37 degree C. water for 1 day, and then cut to a length of 7 mm. Compressive strength was determined according to ISO Standard 7489 using an INSTRON™ universal tester (Instron Corp.) operated at a crosshead speed of 1 mm/min. Results were reported in megapascals (MPa).

### Test Method III—Viscosity

Rheological properties were measured on TA instrument AR G2 at room temperature with simple shear mood. Viscosities of different pastes at shear rate 20 s⁻¹ were used for demonstration of balanced viscosities of different pastes

Test Method IV—Crown Removal, Ease of Cleanup after Placement, and Remnant Cement After Crown Removal

Extracted human molar teeth were potted using acrylic material in a mold and subsequently were prepared using a dental hand-piece by a consultant dentist. Teeth were cleaned by brass brush and polished by fine pumice and stored in DI water prior to crown seating. Protemp Plus System crowns (3M ESPE) were made per manufacturer's instructions and made against corresponding tooth. After crown preparation, cement was used per instructions to seat the crowns and the assembly was put in 37 C oven for the specified clean-up/set time, after which the excess cement was removed (while the ease of clean-up was noted) by a dental explorer, and the assembly was put in 37 C DI water for 1 week. At crown removal session, the dentist used a hemostat to grab on the crown per clinical conditions, and assessed the ease of crown removal on a scale from 1 to 5, from easy to hard. At the same time, notes were made regarding the percentage of the cement left on tooth vs retained inside the crown. Any removal score of 3 and less, and a remnant cement of 5% or less on tooth, were deemed acceptable from removal point of view.

### Preparation of Pastes

Paste A was prepared by adding HEMA or PEGDMA, DI water, HPC, ATU, and DMAPE in a mixing cup, and speed mixing on a Speed Mixer (from FlackTek Inc, Landrum, S.C.) to form a clear solution. The remaining components were then added according to the formulation, followed by speed mixing at 300 rpm for 2 minutes. Paste mixing uniformity was checked, and, if necessary, mixing was continued at the same rpm until a uniform paste A was formed.

Paste B was prepared by adding PEGDMA, BHT, BPO, CPQ, and UDMA into a mixing cup, and speed mixing to form a clear solution. The remaining components were then added according to the formulation, followed by speed mixing at 3000 rpm for 2 minutes. Paste mixing uniformity was checked, and, if necessary, mixing was continued at the same rpm until a uniform paste B was formed.

TABLE 1
Paste A composition (wt %)

| Component | Example 1 | Example 2 | Example 3 |
|---|---|---|---|
| DI Water | 7.80 | 7.80 | 7.80 |
| HPC | 1.79 | 1.79 | 1.79 |
| HEMA | 10.13 | 10.13 | 0.00 |
| PEGDMA 400 | 0.00 | 0.00 | 10.13 |
| ATU | 0.22 | 0.22 | 0.22 |
| DMAPE | 0.55 | 0.22 | 0.55 |
| PEG 300 | 27.34 | 27.34 | 27.34 |
| ST Coarse Glass | 48.59 | 49.06 | 48.59 |
| R812S | 3.58 | 3.58 | 3.58 |

TABLE 2
Paste B composition (wt %)

| Component | Example 1 | Example 2 | Example 3 |
|---|---|---|---|
| PEGDMA 400 | 34.30 | 42.74 | 34.13 |
| BHT | 0.059 | 0.059 | 0.059 |
| BPO | 1.00 | 1.00 | 1.00 |
| PP | 0.00 | 0.00 | 0.50 |
| CPQ | 0.10 | 0.00 | 0.10 |
| UDMA | 23.40 | 15.00 | 23.28 |
| VBCP | 7.50 | 7.50 | 7.46 |
| Zr—Si Filler | 28.94 | 29.00 | 28.80 |
| R812S | 4.70 | 4.70 | 4.68 |

Except for individual viscosity testing, the pastes were loaded into the dispensing equipment described above, then extruded through the automixing tip to provide mixed pastes used for the test methods.

TABLE 3
Testing results

| Test Method | Example 1 | Example 2 | Example 3 |
|---|---|---|---|
| Extrusion Force | 9.8 (0.0) | 7.1 (0.7) | 8.4 (0.1) |
| (lb-f, (standard | | | |
| deviation)) | | | |
| Compressive | 46 (3) | Not tested | 40 (7) |
| Strength MPa | | | |
| (standard | | | |
| deviation) | | | |
| Viscosity of | Not tested | Not tested | 18 |
| paste A, Pa · s @ | | | |
| 20 1/s | | | |
| Viscosity of | Not tested | Not tested | 13 |
| paste B, Pa · s @ | | | |
| 20 1/s | | | |
| Ease of cleanup | Very easy | Very easy | Very easy |
| after placement | | | |
| Crown | 1 | 2 | 1 |
| Removal | | | |
| Remnant cement | 0 | 0 | 0 |
| after crown | | | |
| removal (%) | | | |

What is claimed is:

1. A dental cement composition for forming a temporary bond, comprising:
a first part, the first part including water, an initiator, a reducing agent and a first filler;
a second part, the second part including a monomer having at least one ethylenically unsaturated group per monomer molecule, an oxidizing agent and a second filler; and
an adhesion reducing component of a polyhydric alcohol present in one of the first part or the second part of the dental cement composition for forming the temporary bond.

2. The dental cement composition of claim 1, where the water is present in the first part in an amount of 5 to 20 percent by weight based on the total weight of the first part.

3. The dental cement composition of claim 1, where the first filler and the second filler include a coupling agent.

4. The dental cement composition of claim 1, where at least one of the first filler and the second filler is a nonreactive filler.

5. The dental cement composition of claim 1, where the liquid monomer is selected from the group consisting of 2-hydroxyethyl methacrylate, glycerol monomethacrylate, polyethylene glycol dimethacrylate, sorbitol methacrylate or a combination thereof.

6. The dental cement composition of claim 1, where the adhesion reducing component is selected from the group consisting of glycerol, polyethylene glycol, poly(propylene glycol) or a combination thereof.

7. The dental cement composition of claim 1, where the adhesion reducing component is present in an amount of 1 to 95 percent by weight based on the total weight of the dental cement composition.

8. The dental cement composition of claim 1, where the reducing agent and the oxidizing agent are each present in an amount of 0.01 to 10 percent by weight based on the total weight of the dental cement composition.

9. The dental cement composition of claim 1, where the oxidizing agent is selected from the group selected the group consisting of a persulfate, benzoyl peroxide, a hydroperoxide, a salt of cobalt (III), a salt of iron (III), hydroxylamine, perboric acid, a salt of a permanganate anion or a combination thereof.

10. The dental cement composition of claim 1, where the reducing agent is selected from the group consisting of ascorbic acid, metal complexed ascorbic acid, an aromatic amine, cobalt (II) chloride, ferrous chloride, ferrous sulfate, hydrazine, hydroxylamine, oxalic acid, thiourea, thiourea, an alkyl thiourea, a salt of a dithionite, 1-allyl-2-thiourea, thiosulfate, an aromatic sulfinic acid salt, sulfite anion or a combination thereof.

11. The dental cement composition of claim 1, where the initiator is a visible light photoinitiator.

12. The dental cement composition of claim 1, where a volume ratio of the first part to the second part is from 1.2:1 to 1:1.2.
